(19) United States
(12) Patent Application Publication
(10) Pub. No.: US 2021/0138475 A1
(43) Pub. Date: May 13, 2021

(54) Bottle Holder and Safety Cabinet Having Bottle Holder

(71) Applicant: Hitachi Industrial Equipment Systems Co., Ltd., Chiyoda-ku, Tokyo (JP)

(72) Inventor: Takeshi KANEKO, Tainai (JP)

(21) Appl. No.: 16/623,595

(22) PCT Filed: Feb. 4, 2019
(86) PCT No.: PCT/JP2019/003894

§ 371 Date: Dec. 17, 2019

(30) Foreign Application Priority Data
Apr. 24, 2018 (JP) 2018-083424

(51) Int. Cl. B01L 9/00; B01L 1/02; B01L 7/00

(52) U.S. Cl. CPC B01L 9/00; B01L 1/025; B01L 7/00; B01L 2300/1811; B01L 2200/141; B01L 2300/0609; B01L 2200/025

(57) ABSTRACT

An object of the present invention is to provide a bottle holder that is capable of facilitating the exchange of a bottle with a tube, and a biosafety cabinet having the bottle holder. In order to achieve the object, there is provided a bottle holder for a bottle that is used in a biosafety cabinet which has an operation space and is provided with a support rod lying across the operation space in a width direction, the holder including: a side holding portion that holds a side periphery of the bottle; a bottom holding portion that holds the vicinity of a bottle cap of the bottle and has a bottom opening which embraces the bottle cap; and an opening, which continues from the bottom opening to an upper surface, in the bottom holding portion and the side holding portion, in which the holder holds the bottle downward.

### TECHNICAL FIELD

The present invention relates to a biosafety cabinet that eliminates, using an air barrier, a contamination risk from outside.

### BACKGROUND ART

In the related art, in the industrial fields such as the medical field and the pharmaceutical field, a biosafety cabinet is used as a countermeasure against biological hazard. The biosafety cabinet has the isolation capability of being able to protect a specimen from sundry bacteria from outside by providing an air barrier and performing an operation in a partitioned space having an opening portion in a portion thereof.

Patent Document 1 and Patent Document 2 disclose the background art of the technical field. Patent Document 1 discloses a biosafety cabinet that includes a coupling portion connected to an exhaust port of the biosafety cabinet, an opening portion which is different from the coupling portion and into which air flows, and an open type duct having an exhaust duct, the cabinet includes a differential pressure sensor that detects a difference between a pressure in a space where the open type duct is disposed and a pressure in the open type duct, and detection means for outputting a detection signal when an absolute value of the differential pressure sensor becomes less than or equal to a predetermined threshold value. In addition, Patent Document 2 discloses a point that see-through windows are provided in a portion of a back wall or a side wall of an operation space of the biosafety cabinet and in a portion of a main body rear wall or a main body side wall of a biosafety cabinet which is separated from the back wall or the side wall by a circulation flow path, so as to enable an operator to see through both walls, a display device is installed in an outside part of the see-through window, and the operator inserts the arms from a front opening portion of the biosafety cabinet to perform an operation from a front shutter while seeing the operation space.

### CITATION LIST

### Patent Document

Patent Document 1: JP 2017-078527 A

Patent Document 2: JP 2016-165249 A

### SUMMARY OF THE INVENTION

### Problems to be Solved by the Invention

As shown in Patent Document 1 and Patent Document 2, in the biosafety cabinet for safely performing an operation while maintaining an inside purity, there is a case where the operator uses a bottle containing a solution such as purified water, a pharmaceutical solution, or a reagent in the biosafety cabinet. At the time, if the bottle is disposed on an operation stage in the operation space, the operation stage becomes contaminated, which is a concern. Furthermore, an air flow becomes restricted, which is a concern. In addition, the operation space above the operation stage is reduced, which is a problem. For this reason, it is conceivable to hold the bottle in the air via a bottle holder; however, at the time, a bottle holder which is capable of facilitating the exchange of the bottle has not been considered.

The present invention is made in light of the problems, and an object of the present invention is to provide a bottle holder that is capable of facilitating the exchange of a bottle, and a biosafety cabinet having a bottle holder.

### Solutions to Problems

The present invention is made in light of the background art and the problems, and as an example of the present invention, there is provided a bottle holder for a bottle that is used in a biosafety cabinet which has an operation space and is provided with a support rod lying across the operation space in a width direction, the holder including: a side holding portion that holds a side periphery of the bottle; a bottom holding portion that holds the vicinity of a bottle cap of the bottle and has a bottom opening which embraces the bottle cap; and an opening, which continues from the bottom opening to an upper surface, in the bottom holding portion and the side holding portion, in which the holder holds the bottle downward.

### Effects of the Invention

According to the present invention, it is possible to provide a bottle holder that is capable of facilitating the exchange of a bottle, and a biosafety cabinet having a bottle holder.

### BRIEF DESCRIPTION OF THE DRAWINGS

FIG. 1 is a schematic front view of a biosafety cabinet in the related art.

FIG. 2 is a schematic side view of the biosafety cabinet when a cross section A-A′ in FIG. 1 is seen from right.

FIG. 3 is a view illustrating an air flow when the biosafety cabinet in FIG. 2 operates.

FIG. 4 is a configuration view of a bottle with a tube in Example 1.

FIG. 5 is a schematic configuration view of a bottle holder in Example 1.

FIG. 6 is a schematic configuration view of a bottle holder in Example 2.

FIG. 7 is a view illustrating a state where the bottle is held with a support rod in Example 2.

FIG. 8 is a schematic configuration view of a bottle and a bottle holder in Example 3.

FIG. 9 is a specific configuration view of a bottle holder in Example 5.

FIG. 10 is a specific configuration view of a bottle holder in Example 6.

### MODE FOR CARRYING OUT THE INVENTION

Hereinafter, examples of the present invention will be described with reference to the drawings.

### Example 1

First of all, initially, a biosafety cabinet in the related art which is the premise of the present example will be described.

FIG. 1 illustrates a schematic front view of the biosafety cabinet. In addition, FIG. 2 illustrates a schematic side view of the biosafety cabinet when a cross section A-A′ in FIG. 1 is seen from right.

An opening is provided in a central area of a housing 101 of a biosafety cabinet 100, and an operation space 104 is provided therebehind. A front plate 102 is provided on a front surface side of the operation space 104 so as to block an upper portion of the opening, an operation opening 103 is provided on a lower side thereof, and an operator inserts the hands into the operation space 104 from the operation opening 103 to perform an operation. The front plate 102 is formed of a transparent material such as glass, and the operator can see an operation through the front plate. In addition, a support rod 20 lies across the operation space 104 in a width direction, and a specimen, an instrument, or the like including an infusion solution bag which is used in the operation space can be suspended thereon.

An operation stage 105 which is substantially planar is provided on a bottom surface of the operation space 104, and the operator performs an operation on the operation stage. An air intake port 107 leading downward is provided close to the operation opening 103 on a front side of the operation stage 105. The air intake port 107 is formed of, for example, a slit that extends along the operation opening 103 in a rightward and leftward direction of the housing. A back flow path 108 leading from the air intake port 107 to an upper portion of the housing is provided on a back surface side of the operation space 104.

An air blow side fan filter unit (FFU) 109 is provided above the operation space 104. The air blow side FFU 109 is formed of a fan which is rotated by a motor, and a filter, for example, a HEPA filter 109A which removes microparticles, and blows purified air, from which the microparticles are removed, into the operation space 104. An exhaust side fan filter unit (FFU) 110 is provided in the upper portion of the housing 101, and removes microparticles from a portion of air with a filter, for example, a HEPA filter 110A to exhaust the portion of air outside the device.

In FIG. 3, an air flow when the biosafety cabinet operates is illustrated with the arrows. An air flow 90 which is taken in from the air intake port 107 on a front surface side of the operation stage 105 is blown, as shown with reference sign 91, into the operation space 104 from the air blow side FFU 109 through a lower portion of the housing, the back flow path 108, and the upper portion of the housing. Since purified air from which microparticles are removed by the HEPA filter 109A of the air blow side FFU 109 is blown into the operation space 104, the operation space 104 is maintained in a purified state. At the time, if only an air flow denoted by reference sign 92 is blown into the operation space 104, air in the operation space leaks outside, which is a concern. For this reason, the exhaust side FFU 110 is provided, and a portion of air is discharged outside through the HEPA filter 110A. Therefore, a pressure in the operation space decreases, and an air flow 94 to be introduced from outside to inside through the operation opening 103 in a lower part of the front plate 102 is formed. If the air flow 94 flows directly into the operation space, the purity of the operation space decreases. However, since all of the air flow 94 flowing in from the operation opening 103 and the majority of the air flow 92 blown into the operation space are taken in from the air intake port 107 by properly controlling the air volume of the air flow 92 which is blown into the operation space from the air blow side FFU 109, and the air volume of an air flow 93 which is exhausted outside from the exhaust side FFU 110, owing to the air flow 92 blown into the operation space 104, an atmospheric barrier (air barrier) is formed to prevent the air flow 94 from flowing into the operation space 104 from the operation opening 103. Therefore, it is possible to realize an equilibrium where the operation space 104 is not contaminated by air from outside and no air leaks outside before the inside is purified.

Therefore, even though the operator inserts the hands into the operation space 104 through the operation opening 103 to perform an operation, it is possible to realize the maintenance of the purity and the prevention of contamination.

Here, there is a case where the operator uses a bottle containing a solution such as purified water, a pharmaceutical solution, or a reagent in the biosafety cabinet. At the time, if the bottle is disposed on the operation stage in the operation space, the operation stage becomes contaminated, which is a concern. Furthermore, an air flow becomes restricted, which is a concern. In addition, the operation space above the operation stage becomes small, which is a problem. Then, it is conceivable to suspend, via a bottle holder, the bottle on the support rod on which a specimen, an instrument, or the like including an infusion solution bag which is used in the operation space can be suspended and which lies across the operation space in the width direction; however, merely if the bottle is suspended, the solution does not naturally come out from the bottle, which a problem. Then, it is configured that a tube is attached to the bottle, the bottle is held in the air with the tube facing downward, a cock is provided in the middle of the tube, and the solution in the bottle can be injected into the specimen by opening the cock when necessary.

In the present example, hereinafter, a bottle holder which is capable of facilitating the exchange of the bottle with a tube will be described.

FIG. 4 is a configuration view of a bottle with a tube in the present example. In FIG. 4, 30 is a bottle main body, 31 is a bottle cap, and 32 is a tube extending from the bottle cap 31. In addition, 33 is a label that is affixed to the bottle main body to disclose contents of the bottle. In the present example, the bottle has a quadrangular column shape; however, the present invention is not limited thereto. Incidentally, a cock provided in the middle of the tube is not illustrated.

FIG. 5 is a schematic configuration view of a bottle holder in the present example. In FIG. 5, (a) is a front view, (b) is a top view, and (c) is a view illustrating a state where the bottle is mounted on the bottle holder. In FIG. 5, 41 is a front holding portion, 42 is a bottom holding portion, 43 is a rear holding portion, 44R is a right side holding portion, and 44L is a left side holding portion. Namely, the front holding portion 41, the rear holding portion 43, the right side holding portion 44R, and the left side holding portion 44L are side holding portions which hold the side periphery of the bottle.

As illustrated in FIG. 5, the bottom holding portion 42 does not cover all of a bottom surface of the bottle holder, has a bottom opening 46 for embracing at least the bottle cap 31 so as to hold the bottle cap and the tube of the bottle with a tube downward, and holds the vicinity of the bottle cap. Furthermore, the front holding portion 41 is provided with an opening 45 which continues from the bottom surface to an upper surface. Namely, if the bottom opening 46 is provided only in the bottom holding portion 42 and the opening 45 is not provided in the front holding portion 41, when the bottle with a tube is exchanged from the bottle holder, if the bottle is not lifted to the full length of the tube over the bottle holder, the front holding portion 41 is in the way, and thus it is not possible to exchange the bottle with a tube.

As described above, the bottle holder in the present example has the opening 45 that continues from the bottom opening 46 to the upper surface in the bottom holding portion 42 and the front holding portion 41. Therefore, even though there is a tube, it is possible to easily exchange the bottle. When exchanging the bottle with a tube, it is possible to realize a simple operation of temporarily stopping the solution from flowing with the cock provided in the middle of the tube, replacing only the bottle, and continuing to reuse the bottle with a tube. In addition, the opening 45 also provides the effect of being able to easily read the label 33 which discloses the contents of the bottle.

Incidentally, in FIG. 5, in the bottom holding portion 42 and the front holding portion 41, two members are separated from each other to form an opening; however, as long as the bottle can be held, only one side may be provided. In addition, the bottle holder in the present example is not limited to the configuration in FIG. 5. As long as the concept of the holder with the opening continuing from the bottom surface to the upper surface is maintained, the configuration of the bottle holder may be changed in various forms depending on the shape of the bottle.

As described above, according to the present example, it is possible to provide the bottle holder that is capable of facilitating the exchange of the bottle with a tube.

### Example 2

In the present example, a configuration where a bottle holder is provided with a hook portion onto which a hook for suspending on the support rod is to be hung will be described.

FIG. 6 is a schematic configuration view of a bottle holder in the present example. In FIG. 6, (a) is a front view and (b) is a top view. In FIG. 6, the same reference signs are assigned to configuration elements having the same functions as those of the configuration elements in FIG. 5, and descriptions thereof will be omitted. In FIG. 6, the point of difference from FIG. 5 is that a hook portion 47 is provided. Namely, in FIG. 6, the hook portion 47 is provided in each of the right side holding portion 44R and the left side holding portion 44L.

FIG. 7 illustrates a state where the bottle is held with the support rod 20. In FIG. 7, (a) illustrates a state where the bottle holder is held with the support rod 20 and the bottle is suspended, and (b) illustrates the support rod 20 and a hook 48 which is an accessory thereto. The hook 48 with a non-detachable structure is movably coupled to the support rod 20, and has a U-shaped part with a U shape positioned opposite to the location of coupling to the support rod 20. It is possible to suspend the bottle holder and the bottle on the support rod 20 via the hook 48 coupled to the support rod 20 by hanging the U-shaped part of the hook 48 onto the hook portion 47.

As described above, according to the present example, it is possible to provide the bottle holder that is capable of facilitating the exchange of the bottle with a tube, and a biosafety cabinet having the bottle holder.

### Example 3

In the present example, a bottle holder for a bottle which is taper-shaped on a bottle cap side will be described.

FIG. 8 is a schematic configuration view of a bottle and a bottle holder in the present example. In FIG. 8, (a) is a front view of the bottle and (b) is a front view of the bottle holder. In FIG. 8, the same reference signs are assigned to configuration elements having the same functions as those of the configuration elements in FIG. 6, and descriptions thereof will be omitted. In FIG. 8, the point of difference from FIG. 6 is that the bottle has a taper portion 34 on the bottle cap side and correspondingly, also the bottom holding portion 42 of the bottle holder is shaped to be inclined along the taper portion 34. Incidentally, in FIG. 8, the bottom holding portion 42 is formed in such a manner as to extend from each of the right side holding portion 44R and the left side holding portion 44L; however, the present invention is not limited thereto, and the bottom holding portion 42 may be formed in such a manner as to extend from the rear holding portion 43.

As described above, according to the present example, it is possible to provide the bottle holder that is capable of reliably holding also the bottle with a taper.

### Example 4

In the present example, a configuration where a bottle holder is provided with a temperature regulation mechanism will be described.

If there is a temperature difference between a specimen and a solution in the bottle, when the solution in the bottle is injected into the specimen, there is a possibility of causing a temperature difference in the specimen and generating a temperature history in the cultured cells or the like. In order to prevent the possibility, in the present example, a bottle holder is provided with a temperature regulation mechanism. In particular, since in many cases, it is desirable to raise the temperature higher than a room temperature when performing an operation in the biosafety cabinet, a heating mechanism is provided as the temperature regulation mechanism. Various means such as a nichrome wire heater and a ceramic heater can be applied as the heating mechanism. A configuration view in the present example is not illustrated; however, it is possible to heat the bottle holder and to heat the solution in the bottle held with the bottle holder by adding the heating mechanism at a random location on the bottle holder. Incidentally, the bottle holder is preferably made of stainless steel due to the ease to raise the temperature and the ease to maintain a purified state. In addition, the bottle held with the bottle holder may be directly heated instead of heating the bottle holder.

In addition, since it is necessary to supply an electric power to the heating mechanism, it is possible to realize an electric power supply to the heating mechanism of the bottle holder via the support rod 20, for example, by providing an electric power supply wiring integrally with the hook 48 in FIG. 7. Naturally, the electric power supply wiring may be disposed separately from the hook 48; however, also in this case, the electric power supply wiring is preferably disposed close to the hook 48.

As described above, according to the present example, it is possible to provide the bottle holder which when a solution in the bottle is injected into a specimen, is capable of preventing the generation of a temperature history induced by a temperature difference in the specimen.

### Example 5

In the present example, a specific configuration of the bottle holder in Example 3 will be described.

FIG. 9 is a specific configuration view of a bottle holder in the present example. In FIG. 9, the same reference signs are assigned to configuration elements having the same functions as those of the configuration elements in FIG. 8, and descriptions thereof will be omitted.

In FIG. 9, (a) is a top view, (b) is a front view, (c) is a bottom view, (d) is a right side view, and (e) is a perspective view seen diagonally from above. As illustrated in FIG. 9, the bottle holder is formed such that with respect to a bottle with a taper, a taper portion provided close to the bottle cap 31 is held with the bottom holding portion 42 which is inclined, the side periphery of the bottle is held with the front holding portion 41, the rear holding portion 43, the right side holding portion 44R, and the left side holding portion 44L, each of the bottom holding portion 42 and the front holding portion 41 is formed of two separated members and has an opening therebetween, and an opening continuing from a bottom surface to an upper surface is provided.

In addition, the bottle holder has a structure where the hook portion 47 is provided in each of the right side holding portion 44R and the left side holding portion 44L, and the bottle can be suspended on the support rod via the hook 48 coupled to the support rod by hanging the U-shaped part of the hook 48, which is an accessory to the support rod, onto the hook portion 47. Incidentally, the hook 48 is detachable by inserting and removing the hook portion 47 in an opening direction of the U-shaped part of the hook 48.

As described above, according to the present example, it is possible to provide the bottle holder that is capable of facilitating the exchange of the bottle with a tube, and a biosafety cabinet having the bottle holder.

### Example 6

In the present example, a specific configuration of a bottle holder, which corresponds to a bottle where the bottle cap is formed to be inclined rather than facing directly upward, will be described.

FIG. 10 is a specific configuration view of a bottle holder in the present example. In FIG. 10, the same reference signs are assigned to configuration elements having the same functions as those of the configuration elements in FIG. 9, and descriptions thereof will be omitted. In addition, in FIG. 10, similar to FIG. 9, (a) is a top view, (b) is a front view, (c) is a bottom view, (d) is a right side view, and (e) is a perspective view seen diagonally from above.

In FIG. 10, the point of difference from FIG. 9 is that in a bottle, the bottle cap is formed to be inclined rather than facing directly upward. For this reason, as illustrated in FIG. 10(d), when the bottle is suspended, the bottle itself is held in an inclined manner such that the bottle cap faces directly downward. For this reason, a hook part of the U-shaped part of the hook 48 onto the hook portion 47 is configured such that the bottle is held in an inclined manner. In addition, the bottom holding portion 42 is a member extending from the rear holding portion 43, and has the function of holding the bottle from a lower surface with the rear holding portion 43 and the front holding portion 41. Since the bottle holder with the opening continuing from the bottom surface to the upper surface is formed, also the bottle holder in the present example is capable of facilitating the exchange of the bottle with a tube.

As described above, according to the present example, it is possible to provide the bottle holder that is capable of reliably holding the bottle where the bottle cap is formed to be inclined and facilitating the exchange of the bottle with a tube, and a biosafety cabinet having the bottle holder.

The examples have been described above; however, the present invention is not limited to the examples, and includes various modification examples. For example, the examples have been described in detail so as to facilitate the understanding of the present invention, and the present invention is not necessarily limited to including all of the described configurations. In addition, a portion of a configuration of an example can be replaced with a configuration of another example. In addition, a configuration of another example can also be added to a configuration of an example. In addition, the addition, the deletion, or the replacement of another configuration can be made with respect to a portion of a configuration of each example.

### REFERENCE SIGNS LIST

20 Support rod

30 Bottle main body

31 Bottle cap

32 Tube

33 Label

34 Taper portion

41 Front holding portion

42 Bottom holding portion

43 Rear holding portion

44R Right side holding portion

44L Left side holding portion

45 Opening

46 Bottom opening

47 Hook portion

48 Hook

100 Biosafety cabinet

101 Housing

102 Front plate

103 Operation opening

104 Operation space

105, 105T Operation stage

107 Air intake port

108 Back flow path

109 Air blow side fan filter unit (FFU)

109A Air blow side HEPA filter

110 Exhaust side fan filter unit (FFU)

110A Exhaust side HEPA filter

1. A bottle holder for a bottle that is used in a biosafety cabinet which has an operation space and is provided with a support rod lying across the operation space in a width direction, the holder comprising:
a side holding portion that holds a side periphery of the bottle;
a bottom holding portion that holds the vicinity of a bottle cap of the bottle and has a bottom opening which embraces the bottle cap; and
an opening, which continues from the bottom opening to an upper surface, in the bottom holding portion and the side holding portion,
wherein the holder holds the bottle downward.

2. A bottle holder for a bottle that is used in a biosafety cabinet which has an operation space and is provided with a support rod lying across the operation space in a width direction,
wherein the bottle is a bottle with a tube which is formed of a bottle main body, a bottle cap, and a tube extending from the bottle cap, and
wherein the bottle holder has a side holding portion that holds a side periphery of the bottle, a bottom holding portion that holds the vicinity of the bottle cap and has a bottom opening which embraces the bottle cap, and an opening, which continues from the bottom opening to an upper surface, in the bottom holding portion and the side holding portion, and holds the bottle with the bottle cap and the tube facing downward.

3. The bottle holder according to claim 1,
wherein the side holding portion is formed of a front holding portion, a rear holding portion, a right side holding portion, and a left side holding portion, and
wherein each of the bottom holding portion and the front holding portion is formed of two separated members, and has an opening therebetween.

4. The bottle holder according to claim 1,
wherein a hook with a non-detachable structure is movably coupled to the support rod, and
wherein the side holding portion is provided with a hook portion onto which the hook is to be hung.

5. The bottle holder according to claim 1,
wherein the bottom holding portion has an inclined shape.

6. The bottle holder according to claim 1,
wherein a heating mechanism is provided.

7. The bottle holder according to claim 4,
wherein the hook has a U-shaped part positioned opposite to a location of coupling to the support rod, and
wherein the hook is detachable by inserting and removing the hook portion in an opening direction of the U-shaped part.

8. The bottle holder according to claim 4,
wherein the hook portion is structured to hang onto the hook such that the bottle is held in an inclined manner when the bottle is suspended.

9. A biosafety cabinet that is provided with an operation space having an operation stage, a front plate formed in a front surface of the operation space, an operation opening provided in a lower portion of the front plate, an air intake port leading downward which is provided close to the operation opening on a front side of the operation stage, an air circulation path through which air which is taken in from the air intake port flows along a lower portion, a back surface, and an upper portion of the operation space, and a support rod which lies across the operation space in a width direction,
wherein a hook with a non-detachable structure is movably coupled to the support rod,
wherein the biosafety cabinet has a bottle holder that holds a bottle, and
wherein the bottle holder has a side holding portion that holds a side periphery of the bottle, a bottom holding portion that holds the vicinity of a bottle cap of the bottle and has a bottom opening which embraces the bottle cap, an opening, which continues from the bottom opening to an upper surface, in the bottom holding portion and the side holding portion, and a hook portion which is provided in the side holding portion and onto which the hook is to be hung, and holds the bottle downward.

10. A biosafety cabinet that is provided with an operation space having an operation stage, a front plate formed in a front surface of the operation space, an operation opening provided in a lower portion of the front plate, an air intake port leading downward which is provided close to the operation opening on a front side of the operation stage, an air circulation path through which air which is taken in from the air intake port flows along a lower portion, a back surface, and an upper portion of the operation space, and a support rod which lies across the operation space in a width direction,
wherein a hook with a non-detachable structure is movably coupled to the support rod,
wherein the biosafety cabinet has a bottle holder that holds a bottle with a tube which is formed of a bottle main body, a bottle cap, and a tube extending from the bottle cap, and
wherein the bottle holder has a side holding portion that holds a side periphery of the bottle, a bottom holding portion that holds the vicinity of the bottle cap and has a bottom opening which embraces the bottle cap, an opening, which continues from the bottom opening to an upper surface, in the bottom holding portion and the side holding portion, and a hook portion which is provided in the side holding portion and onto which the hook is to be hung, and holds the bottle with the bottle cap and the tube facing downward.

11. The biosafety cabinet according to claim 9,
wherein the bottle holder has a heating mechanism, and
wherein an electric power supply wiring to the heating mechanism is provided integrally with the hook, and an electric power is supplied to the heating mechanism via the support rod.
